(19) United States
(12) Patent Application Publication
(10) Pub. No.: US 2009/0117192 A1
(43) Pub. Date: May 7, 2009

(54) COMPOSITION FOR TRANSDERMAL ABSORPTION AND FORMULATION COMPRISING A POLYMERIC MATRIX FORMED THEREFROM

(76) Inventors: Young Chang Ah, Yongin-si (KR); Byoung Young Woo, Yongin-si (KR); Yang Gyu Choi, Yongin-si (KR); Han Moe Ki, Yongin-si (KR)

Correspondence Address: ROTHWELL, FIGG, ERNST & MANBECK, P.C., 1425 K STREET, N.W., SUITE 800, WASHINGTON, DC, 20005, US

(21) Appl. No.: 12/300,213

(22) PCT Filed: May 3, 2007
(86) PCT No.: PCT/KR07/02201

§ 371 Date: Nov. 10, 2008

(51) Int. Cl. A61K 9/10; A61K 31/5415; A61K 31/60

(52) U.S. Cl. 424/487; 514/226.5; 514/159; 424/484; 424/486

(57) ABSTRACT

The inventive composition for transdermal absorption characterized by comprising a nonsteroidal anti-inflammatory drug as an active ingredient together with an alkali metal-containing alcohol derivative as a solubilizing agent for the active ingredient comprises a high concentration of the active ingredient while using only a small amount of the solvent, and a transdermal absorption formulation comprising a polymeric matrix formed from the composition is capable of maximizing the skin absorption of the active ingredient with minimal skin irritation.

### FIELD OF THE INVENTION

The present invention relates to a composition for transdermal absorption capable of retaining a high concentration of a nonsteroidal anti-inflammatory drug as an active ingredient; and a transdermal absorption formulation comprising a polymeric matrix derived therefrom, which can maximize the skin absorption of the active ingredient.

### BACKGROUND OF THE INVENTION

Nonsteroidal anti-inflammatory drugs have been widely used as therapeutic agents for the inflammation or pain caused by acute arthritis, chronic arthritis, myalgia, spondylitis, osteoarthritis or the like. However, such nonsteroidal anti-inflammatory drugs may cause various side effects after oral administration (Catherine A. Heyneman et al., Drugs, pp. 555˜574, 2000), and, accordingly, such drugs are mainly delivered through the skin.

A dosage form employing a delivery system of an active ingredient through the skin is generally called a transdermal absorption formulation. In order to deliver a sufficient amount of the active ingredient through the skin, methods to enhance the skin absorption into the outermost layer of the skin, namely the stratum corneum must be developed. Thus, methods of increasing the solubility of the active ingredient in the composition by using a solvent for the active ingredient together with the active ingredient have been disclosed.

For example, Korean Patent Publication Nos. 1993-32006 and 2003-80982 disclose a method of employing alkanolamine as a solvent for the active ingredient and a nonprotonic organic solvent such as dimethylsolfoxide as a solubilizing agent; Japanese Patent Publication No. 2003-323310 disclose a method of using a mixture of a mint ingredient with alkanolamine or alkanolamide as a solvent for the active ingredient; and Korean Patent Publication No. 2001-36079 disclose the use of a composition in the form of a salt prepared by treating alkanolamine with nonsteroidal anti-inflammatory drug.

However, the above-mentioned methods are likely to cause skin irritation due to the use of the excessive amount of the solvent for the active ingredient, e.g., alkanolamine and alkanolamide, which reaches up to 40% weight based on the total weight of the composition.

In addition, a method using a strong basic compound (hydroxide-releasing compound) such as metal hydroxides or ammonia (U.S. Pat. Nos. 6,586,000, 6,645,520 and 6,835,392) has been suggested for increasing the solubility of the active ingredient in a composition. However, the miscibility thereof with a conventional polymeric adhesive used together with the strong base-active ingredient mixture is poor due to the presence of water used for dissolving the strong basic compound, which leads to the unsatisfactory workability and stability of the dosage form.

### SUMMARY OF THE INVENTION

Accordingly, it is an object of the present invention to provide a composition for transdermal absorption comprising a solubilizing agent for the active ingredient capable of significantly increasing the solubility of nonsteroidal anti-inflammatory drug as the active ingredient, a small amount of the solubilizing agent being capable of dissolving a high concentration of an active ingredient with minimal skin irritation.

It is another object of the present invention to provide a transdermal absorption formulation comprising a polymeric matrix prepared using the composition for transdermal absorption. The transdermal absorption formulation can maximize the skin absorption of the active ingredient.

In accordance with one aspect of the present invention, there is provided a composition for transdermal absorption comprising a nonsteroidal anti-inflammatory drug, an alkali metal-containing alcohol derivative, a polymeric adhesive and a volatile solvent.

In accordance with one aspect of the present invention, there is provided a transdermal absorption formulation comprising a release paper; a polymeric matrix prepared from the composition for transdermal absorption; and a supporting substrate stacked together in that order.

### BRIEF DESCRIPTION OF DRAWINGS

The above and other objects and features of the present invention will become apparent from the following description of the invention, when taken in conjunction with the accompanying drawings:

FIGS. 1A, 1B and 1C are longitudinal cross-sectional views of various types of transdermal absorption formulations comprising a polymeric matrix formed from the inventive composition for transdermal absorption.

### BRIEF DESCRIPTION OF THE REFERENCE NUMERALS IN DRAWINGS

| | |
|---|---|
| 1: supporting substrate | 2: polymeric matrix |
| 3: release paper | 4: supporting substrate adhesive layer |
| 5: skin adhesive layer | |

### DETAILED DESCRIPTION OF THE INVENTION

The inventive composition for transdermal absorption comprising poorly water-soluble nonsteroidal anti-inflammatory drug as an active ingredient is characterized by comprising alkali metal-containing alcohol derivatives as a solubilizing agent for the active ingredient.

The inventive composition for transdermal absorption comprises a nonsteroidal anti-inflammatory drug, an alkali metal-containing alcohol derivative and a polymeric adhesive, the amounts of which range from 0.1 to 50% by weight, 0.01 to 10% by weight, and 40 to 98% by weight, respectively, based on the total weight of the solids.

Each ingredient of the inventive composition for transdermal absorption is described in detail as follows:

### <Nonsteroidal Anti-Inflammatory Drug>

In the present invention, representative examples of the nonsteroidal anti-inflammatory drug used as the active ingredient include salicylic acid, celecoxib, rofecoxib, meloxicam, tenoxicam, isoxicam, piroxicam, ketoprofen, ketorolac, flurbiprofen, fenoprofen, naproxen, indomethacin, aceclofenac, diclofenac, aspirin, ibuprofen and a mixture thereof.

### <Alkali Metal-Containing Alcohol Derivative>

In the present invention, the alkali metal-containing alcohol derivative used as a solubilizing agent for the active ingredient can significantly increase the solubility of the active ingredient in the composition by changing the polarity of the active ingredient instead of altering the chemical structure of the active ingredient.

The alkali metal-containing alcohol derivative may be used in an amount ranging from 0.01 to 10% by weight, preferably from 0.015 to 4.5% by weight based on the total weight of the solid.

A suitable alkali metal-containing alcohol derivative that can be used in the present invention is shown in formula (I), and preferable examples thereof preferably include sodium methoxide, sodium ethoxide, sodium propoxide and a mixture thereof:

M—O—R (I)

wherein,
M is alkali metal, O is oxygen and R is C₁₋₅ alkyl.

### <Polymeric Adhesive>

In the present invention, the polymeric adhesive may be an acrylic adhesive or a rubber-based adhesive. The acrylic adhesive is preferably obtained by polymerizing one or more monomers selected from the group consisting of: acrylic monomer having C₁₋₁₈ alkyl group (e.g. methylmethacrylate, ethylmethacrylate, propylmethacrylate, butylmethacrylate, pentylmethacrylate, hexylmethacrylate, heptylmethacrylate, octylmethacrylate, nonylmethacrylate, decylmethacrylate, undecylmethacrylate, dodecylmethacrylate and tridecylmethacrylate); acrylic monomer having an acrylic acid, methacrylic acid or hydroxyl group; and vinylpyrrolidone. The rubber-based adhesive is preferably one or more polymer selected from the group consisting of natural rubber, styrene copolymer, polyisobutylene, polyisoprene and polybutadiene.

### <Volatile Solvent>

Representative examples of the volatile solvent used in the present invention include water, methanol, ethylacetate, tetrahydrofurane, acetone, isopropanol and a mixture thereof. The amount of the volatile solvent does not limit its scope, and in case of an alcohol, it may be used in an amount sufficient to prevent the precipitation of the polymeric adhesive.

### <Other Additives>

The inventive composition for transdermal absorption may further comprise a conventional co-solubilizing agent, a penetration enhancer and a property controlling agent, the amounts of which range from 0.1 to 20% by weight, 0.1 to 20% by weight and 0.1 to 10% by weight based on the total weight of the solids, respectively.

The co-solubilizing agent which serves to increase the efficiency of the alkali metal-containing alcohol derivative may be a polar aprotic organic solvent. Representative examples of the polar aprotic organic solvent include dimethylsulfoxide, dimethylacetamide, dimethylformamide, N-methylpyrrolidone, dimethylisosorbide, isosorbide and a mixture thereof.

Representative examples of the penetration enhancers used in the present invention include fatty acids such as linoleic acid, oleic acid, palmitic acid, stearic acid, capric acid and myristic acid; polyhydric alcohols such as propyleneglycol, polyethyleneglycol, dipropyleneglycol, diethyleneglycol and glycerol; surfactants such as Span® 80, Tween® 80, Labrasol® and Cremophor®; fatty acid alcohol such as oleyl alcohol and stearyl alcohol; fatty acid ester such as isopropyl myristate, propyleneglycol caprylate, glyceryl monolaurate, propyleneglycol laurate and polyethyleneglycol laurate; polyoxyethylene alkylether such as polyoxyethylene laurylether, polyoxyethylene cetylether, polyoxyethylene stearylether, polyoxyethylene oleylether, polyoxyethylene behenylether, polyoxyethylene hexyldecylether and polyoxyethylene decyltetradecylether; alkanolamine such as monoethanolamine, diethanolamine, triethanolamine, isopropanolamine, diisopropanolamine and triisopropanolamine; alkanolamide such as lauryl dimethanolamide; and a mixture thereof.

Representative examples of the property controlling agent used for enhancing the coating convenience and the stability of the dosage form after drying include polyvinylpyrrolidone, polyethyleneoxide, silicon oxide, aluminum oxide, titanium oxide and a mixture thereof.

In accordance with the present invention, the composition for transdermal absorption may be prepared by adding the nonsteroidal anti-inflammatory drug, alkali metal-containing alcohol derivative, polymeric adhesive, and, if necessary, polar aprotic organic solvent as a co-solubilizing agent, penetration enhancers and property controlling agent to a volatile solvent and homogeneously stirring the mixture. The polymeric adhesive is commercially available in the form of a solution in a volatile solvent. When a solid polymeric adhesive is used, it is dissolved in a volatile solvent and mixed with the other components.

In accordance with the present invention, the composition for transdermal absorption thus obtained may be coated on a release paper, the volatile solvent is removed by means of a solvent evaporation method and dried to obtain a polymeric matrix containing the active ingredient. The drying process may be carried out at 60 to 90° C. for 10 to 30 hours. The polymeric matrix thus obtained has a self-adhesive property and its thickness ranges from 10 to 120 μm.

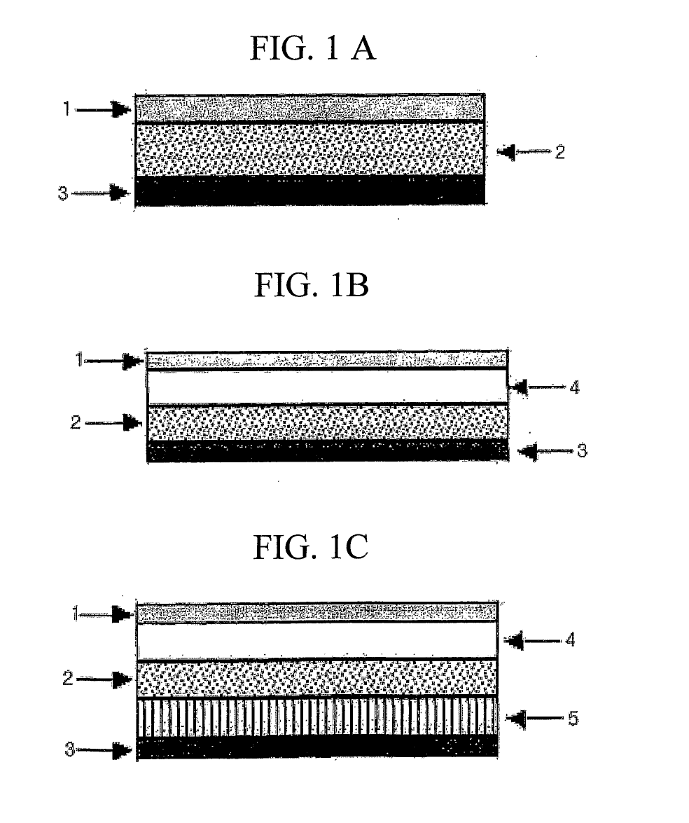

Subsequently, the polymeric matrix formed on the release paper may be combined with a supporting substrate to obtain the inventive transdermal absorption formulation comprising the release paper, polymeric matrix and supporting substrate in that order. Various types of transdermal absorption formulation according to the present invention are shown in FIGS. 1A, 1B and 1C.

The inventive transdermal absorption formulation may further comprise a supporting substrate adhesive layer (tie-layer) 4 disposed between the supporting substrate 1 and the polymeric matrix 2 for improving the adhesive property of the supporting substrate 1 and the polymeric matrix 2, and may further comprise a skin adhesive layer 5 disposed between the release paper 5 and the polymeric matrix 2 for increasing the adhesive property with the skin and adhesion time.

The supporting substrate 1 used in the inventive transdermal absorption formulation to keep the shape of the transdermal absorption formulation, to prevent the loss of the active ingredient and to improve the convenience, may be one or more films made from a material selected from the group consisting of polyolefin (e.g., polyethylene, polypropylene), polyester (e.g., polyethylene terephthalate), polyurethane and aluminum, or one or more fabrics or nonwovens made from a material selected from the group consisting of polyolefin, polyester, nylon and cotton.

The release paper 3 may be a paper or a polymer film coated with silicon or a hydrofluorocarbon resin.

The skin adhesive layer 5 and the supporting substrate adhesive layer 4 may each independently comprise a polymeric adhesive and an adhesive performance improving agent in an amount of 70 to 99% by weight and 1 to 30% by weight.

The polymeric adhesive consisting of the skin adhesive layer 5 and the supporting substrate adhesive layer 4 may be an acrylic adhesive or rubber-based adhesive which may be used in the composition for transdermal absorption. The adhesive performance improving agent may be selected from the group consisting of fatty acid ester such as isopropyl myristate, propyleneglycol caprylate, propyleneglycol laurate, polyethyleneglycol laurate and propyleneglycol oleate; polyethyleneglycol; polyethyleneglycol-polypropyleneglycol copolymer; polyvinylpyrrolidone; inorganic filler such as silicon oxide, aluminum oxide, and titanium oxide; and a mixture thereof. The skin adhesive layer and the supporting substrate adhesive layer can be prepared in accordance with a method similar to that for the preparation of the polymeric matrix, and each layer has a thickness ranging from 10 to 100 μm.

As described above, the inventive transdermal absorption formulation comprising the polymeric matrix formed from the composition containing high concentration of the nonsteroidal anti-inflammatory drug is capable of maximizing the skin absorption of the nonsteroidal anti-inflammatory drug with minimal skin irritation.

The following Examples are given for the purpose of illustration only, and are not intended to limit the scope of the invention.

### EXAMPLES

### Meloxicam Solubility Enhancement Test of Sodium Methoxide

### Examples 1 to 5

Meloxicam was mixed with an equivalent amount of sodium methoxide, and the mixture was dissolved to the saturation point in distilled water, methanol, ethylacetate, tetrahydrofuran or N-methylpyrrolidone. The resulting solution was subjected to high performance liquid chromatography to measure the meloxicam solubility. The results obtained with various solvents, Examples 1 to 5, are shown in Table 1.

### Comparative Examples 1 to 5

Only Meloxicam without using sodium methoxide was dissolved to the saturation point in distilled water, methanol, ethylacetate, tetrahydrofuran or N-methylpyrrolidone, and then the resulting solution was subjected to high performance liquid chromatography to measure the meloxica solubility. The results, Comparative Examples 1 to 5, are shown in Table 1.

TABLE 1

| | | Meloxicam |
|---|---|---|
| | | solubility |
| | Solvents | (mg/Ml) |
| Example 1 | Distilled water | 0.95 |
| Comparative Example 1 | | 0.06 |
| Example 2 | Methanol | 306 |
| Comparative Example 2 | | 0.43 |
| Example 3 | Ethylacetate | 4.95 |
| Comparative Example 3 | | 1.28 |
| Example 4 | Tetrahydrofuran | 67.87 |
| Comparative Example 4 | | 21.91 |
| Example 5 | N-methylpyrrolidone | 490.3 |
| Comparative Example 5 | | 92.7 |

As shown in Table 1, Examples 1 to 5 which meloxicam was dissolved together with sodium methoxide exhibit much higher meloxicam solubilities than those obtained with meloxicam alone Comparative Examples 1 to 5.

### Preparation Example

### Preparation of 2-ethylhexylacrylate vinylpyrrolidone Copolymer as a Polymeric Adhesive

302 g of 2-hexylethylacrylate monomer, 98.0 g of vinylpyrrolidone monomer and 300 g of ethylacetate were added to a three-neck flask equipped with a reflux condenser. The mixture was heated to 60° C. under a nitrogen atmosphere. A mixture of lauroyl peroxide and ethylacetate was added dropwise thereto while maintaining the reaction mixture at 60° C. for 32 hrs. After completion of the reaction, the resulting mixture was cooled and diluted with tetrahydrofuran to obtain a 2-ethylhexylacrylate vinylpyrrolidone copolymer adhesive solution(solid content 16% by weight).

### Composition for Transdermal Absorption and Transdermal Absorption Formulation Comprising a Polymeric Matrix Formed Therefrom

### Example 6

1.5 g of meloxicam (an active ingredient), the 2-ethylhexylacrylate vinylpyrrolidone copolymer solution prepared in Preparation Example in an amount corresponding to 8.5 g of the polymer (a polymeric adhesive), and 0.23 g of sodium methoxide (a solubilizing agent of the active ingredient) were added to 8 g of methanol, and the mixture was homogeneously mixed. The liquid mixture (composition for transdermal absorption) was kept at room temperature until the air bubbles therein were completely removed. Subsequently, the liquid mixture was coated on a release paper having a silicon coat (thickness: 75 μm), and dried at 80° C. for 10 min to obtain a polymeric matrix layer having a thickness of 60 μm coated on the release paper, which was then combined with a polyethylene terephthalate film substrate having a thickness of 25 μm, to obtain a transdermal absorption formulation comprising the release paper, the polymeric matrix and the substrate, in that order.

### Examples 7 to 14 and Comparative Examples 6 to 8

A transdermal absorption formulation was prepared by repeating the procedure of Example 6 except for changing the ingredients and the amounts of the active ingredient, polymeric adhesive, and solubilizing agent of the active ingredient, together with a skin permeation enhancer as shown in Table 2.

### Example 15

A transdermal absorption formulation was prepared by repeating the procedure of Example 6 except for changing the ingredients and the amounts of the active ingredient, polymeric adhesive, and solubilizing agent of the active ingredient, together with a skin permeation enhancer as shown in Table 2, while using a polyester fabric film having a thickness of 400 μm instead of the polyethylene terephthalate as the substrate.

### Example 16

A transdermal absorption formulation was prepared by repeating the procedure of Example 15 except for using a polyisobutylene film having a thickness of 50 μm instead of the polyester fabric, which was coated on a polyester fabric substrate having a thickness of 40 μm.

### Test Example 1

### In vitro Skin Penetration Test of Active Ingredient

The transdermal absorption formulations prepared in Examples 6 to 16, Comparative Examples 6 to 8, and Piroxicam transdermal absorption formulation Trast (Trast®, SK PHARMACEUTICALS) as a Comparative formulation were subjected to in vitro skin penetration tests (skin permeation amount of active ingredient) of the active ingredient.

The in vitro skin penetration test was conducted using Franz diffusion cell. The skin of hairless mice (male, 6 to 7 weeks of age) was excised and cut into 2 cm×2 cm squares, and then the dermal-side of the skin fragment was placed into the receptor of the diffusion apparatus. Subsequently, a transdermal absorption formulation sample was cut into 1.5 cm×1.5 cm squares, the release paper was removed therefrom, and the polymeric matrix was combined and fitted on the exposed percutaneous side of the skin fragment. The receptor of the diffusion apparatus was filled with phosphate buffer (pH 7.4) and the phosphate buffer solution in the receptor was exchanged with a fresh buffer solution at 4, 8 and 24 hrs, while stirring at 600 rpm. The collected phosphate buffer solution was subjected to high performance liquid chromatography (HPLC) and to quantitative analysis to determine the content of the active ingredient in the buffer solution. The quantitative analysis was carried out under the following conditions according to the active ingredients used. The measured active ingredient contents, the amounts of permeated through the skin, are shown Table 2.

### <Meloxicam>

Mobile phase: acetonitril/0.05M sodium acetate solution (pH 3.3)=500/500 (v/v)
Immobile phase: Luna 5μ C18(2)(Phenomenex), 150×4.60 mm, 5μ)
Mobile phase speed: 1 M [symbol] /min
Wave length: UV 355 nm

### <Piloxicam, Tenoxica>

Mobile phase: 0.05M diammonium phosphate/acetonitril/methanol=500/100/400 (v/v/v)
Immobile phase: Luna 5μ C18(2)(Phenomenex), 150×4.60 mm, 5 μ)
Mobile phase speed: 1 M [symbol] /min
Wave length: UV 366 nm

### Test Example 2

### Uniformity of Polymeric Matrix

To examine the uniformity of polymeric matrix prepared in Examples 6 to 16 and Comparative Examples 6 to 8, a dried solid matrix was observed with the naked eye and with an optical microscope. The results are shown in Table 2.

### Test Example 3

### Primary Skin Irritant Test

A phosphate buffer suspension comprising 15% by weight of an equimolar mixture meloxicam and sodium methoxide (pH 7.4) and each of the transdermal absorption formulations prepared in Examples 6 and 7 and Comparative Examples 7 and 8 were subjected to primary skin irritant tests. The test was conducted by using four rabbits (Newzealand Ehite Rabbit) of 6 weeks of age. After removing the hair in the thoracolumbar region of the rabbits, the transdermal absorption formulation was attached hereto for 24 hrs, and the degree of the skin irritation was observed to assess the primary irritation index. The results are shown in Table 3. When a meloxicam suspension was used, the suspension was brought into contact with the thoracolumbar region of the rabbits for 24 hrs.

TABLE 2

| | Ingredients of the polymeric matrix | | | | | Properties | | |
|---|---|---|---|---|---|---|---|---|
| | | | | Co- | | Penetration | Flow | |
| | Active | Adhesive | Solubilizing | solubilizing | Penetration | amount for 24 hrs | for 24 hrs | Matrix |
| | ingredient | (Solid) | agent | agent | enhancer | (μg/cm²) | (μg/cm²/hr) | uniformity* |
| Example | Meloxicam | AVP1 | NaOCH₃ | — | — | 98.7 ± 19.7 | 4.11 ± 0.82 | ◯ |
| 6 | 1.5 g | 8.5 g | 0.23 g | | | | | |
| Example | Meloxicam | AVP1 | NaOCH₃ | — | — | 160.8 ± 15.4 | 6.70 ± 0.64 | ◯ |
| 7 | 3 g | 7 g | 0.46 g | | | | | |
| Example | Meloxicam | AVP1 | NaOCH₃ | NMP | BC 0.5 g | 527.0 ± 77.1 | 21.96 ± 3.21 | ◯ |
| 8 | 1.5 g | 5.5 g | 0.23 g | 2 g | Ole 0.5 g | | | |
| Example | Piroxicam | AVP1 | NaOCH₃ | NMP | BC 0.5 g | 554.0 ± 57.7 | 23.08 ± 2.40 | ◯ |
| 9 | 1.5 g | 5.5 g | 0.24 g | 2 g | Ole 0.5 g | | | |
| Example | Tenoxicam | AVP1 | NaOCH₃ | NMP | BC 0.5 g | 414.6 ± 19.0 | 17.25 ± 0.79 | ◯ |
| 10 | 1.5 g | 5.5 g | 0.24 g | 2 g | Ole 0.5 g | | | |
| Example | Meloxicam | AVP1 | NaOCH₂—CH₃ | NMP | BC 0.5 g | 530.1 ± 56.1 | 22.08 ± 2.33 | ◯ |
| 11 | 1.5 g | 5.5 g | 0.29 g | 2 g | Ole 0.5 g | | | |
| Example | Meloxicam | AVP1 | NaOCH₃ | — | GML 0.5 g | 442 ± 35 | 18.4 ± 1.03 | ◯ |
| 12 | 1.3 g | 6.9 g | 0.10 g | | BC 0.3 g | | | |
| | | | | | DIPA 0.3 g | | | |
| Example | Meloxicam | AVP1 | NaOCH₃ | — | PGML 0.5 g | 300 ± 59 | 12.5 ± 2.45 | ◯ |
| 13 | 1.3 g | 6.9 g | 0.10 g | | BC 0.3 g | | | |
| | | | | | DIPA 0.3 g | | | |
| Example | Meloxicam | AVP1 | NaOCH₃ | — | IPM 0.5 g | 196 ± 26 | 8.2 ± 1.08 | ◯ |
| 14 | 1.3 g | 6.9 g | 0.10 g | | BC 0.3 g | | | |
| | | | | | DIPA 0.5 g | | | |
| Example | Meloxicam | AVP1 | NaOCH₃ | — | BC 0.8 g | 163 ± 39 | 6.7 ± 1.63 | ◯ |
| 15 | 1.3 g | 7.4 g | 0.20 g | | DIPA 0.5 g | | | |
| Example | Meloxicam | AVP1 | NaOCH₃ | — | BC 0.8 g | 472 ± 16 | 19.7 ± 0.67 | ◯ |
| 16 | 1.3 g | 7.4 g | 0.20 g | | DIPA 0.5 g | | | |
| Comparative | Meloxicam | AVP1 | NaOH | — | — | 34.0 ± 8.23 | 1.42 ± 0.34 | ◯ |
| Example 6 | 1.5 g | 8.5 g | 0.18 g | | | | | |
| Comparative | Meloxicam | AVP1 | DIPA | — | — | 105.8 ± 43.8 | 4.40 ± 1.83 | Δ |
| Example 7 | 1.5 g | 8.5 g | 0.57 g | | | | | |
| Comparative | Meloxicam | AVP1 | DIPA | — | — | 128.0 ± 56.7 | 5.33 ± 2.85 | X |
| Example 8 | 3 g | 7 g | 1.14 g | | | | | |

| | | | |
|---|---|---|---|
| Trast ®(SK PARMACEUTICALS PIROXICAM FORMULATION) | 175.0 ± 68.6 | 7.29 ± 2.86 | ◯ |

AVP1: 2-ethylhexylacrylate vinylpyrrolidone
DIPA: diisopropanolamine
NMP: N-methly pyrrolidone
BC: polyoxyethylenecetylether((Nikkol)BC-2, Nikko chemicals Co. Ltd.))
Ole: Polyoxyethyleneoleylehter (Nikkol Oleth-2, Nikko Chemicals Co. Ltd.)
GML: Glycolmonolaurate(Monolurin, Kanto Chemicals Co. Ltd.)
PGML: Propyleneglcolmonolaurate (Emalex PG-M-L, Nihon Emulsion Co. Ltd.)
IPM: Isopropylmyristate (Lexol IPM-NF, Nihon Emulsion Co. Ltd.)
*Matrix uniformity
◯: transparent and uniform appearance
Δ: partially opaque and ununiform appearance
X: whole opaque and ununiform appearance, crystal precipitation

TABLE 3

| | Primary | | |
|---|---|---|---|
| | irritation | | |
| | index(P.I.I.) | Determination | Remarks |
| Meloxicam/sodium | 0.02 | nonirritant | usable |
| methoxide | | | |
| suspension | | | |
| Example 6 | 0.06 | nonirritant | usable |
| Example 7 | 0.06 | nonirritant | usable |
| Comparative | 1.0 | middly irritant | concentration |
| Example 7 | | | control needed |
| Comparative | 1.17 | middly irritant | concentration |
| Example 8 | | | control needed |

*Primary irritation index(P.I.I.)
0 ≦ P.I.I. ≦ 0.5: non-irritant-usable
0.5 ≦ P.I.I. ≦ 2.0: mildly irritant
2.0 ≦ P.I.I. ≦ 5.0: moderately irritant-unusable
5.0 ≦ P.I.I.: severely irritant-unusable

As shown in Table 2, the transdermal absorption formulations prepared in Examples 6 to 16 comprising an alkali metal alkoxide as a solubilizing agent of the active ingredient exhibit highly improved skin permeation of the active ingredient, as compared to Comparative Examples 6 to 8 using other solubilizing agents. On the other hand, the amount of skin permeation of the formulation prepared in Comparative Examples 7 and 8 are similar to those of Examples 6 and 7 comprising same active ingredient. However as shown in Table 3, the formulations of Comparative Examples 7 and 8 induced skin irritation during the primary skin irritant test even when 5% by weight of diisopropanolamin was used. Accordingly, the transdermal absorption formulations of Examples have much reduced skin irritability over those of Comparative Examples.

Further, the commercially available piroxicam formulation Trast exhibits a significantly lower amount of skin permeation, as compared to that of the transdermal absorption formulation prepared in Example 9.

While the embodiments of the subject invention have been described and illustrated, it is obvious that various changes and modifications can be made therein without departing from the spirit of the present invention which should be limited only by the scope of the appended claims.

What is claimed is:

1. A composition for transdermal absorption comprising a nonsteroidal anti-inflammatory drug, an alkali metal-containing alcohol derivative, a polymeric adhesive and a volatile solvent.

2. The composition for transdermal absorption of claim 1, wherein the alkali metal-containing alcohol derivative is a compound of formula (I):
M—O—R (I)
wherein,
M is alkali metal, O is oxygen and R is C₁₋₅ alkyl.

3. The composition for transdermal absorption of claim 2, wherein the alkali metal-containing alcohol derivative is selected from the group consisting of sodium methoxide, sodium ethoxide, sodium propoxide and a mixture thereof.

4. The composition for transdermal absorption of claim 1, which comprises the nonsteroidal anti-inflammatory drug, the alkali metal-containing alcohol derivative and the polymeric adhesive in the amounts of 0.1 to 50% by weight, 0.01 to 10% by weight, and 40 to 98% by weight, respectively, based on the total weight of the solids.

5. The composition for transdermal absorption of claim 1, wherein the nonsteroidal anti-inflammatory drug is selected from the group consisting of salicylic acid, celecoxib, rofecoxib, meloxicam, tenoxicam, isoxicam, piroxicam, ketoprofen, ketorolac, flurbiprofen, fenoprofen, naproxen, indomethacin, aceclofenac, diclofenac, aspirin, ibuprofen and a mixture thereof.

6. The composition for transdermal absorption of claim 1, wherein the polymeric adhesive is an acrylic adhesive or a rubber-based adhesive.

7. The composition for transdermal absorption of claim 6, wherein the acrylic adhesive is obtained by polymerizing a monomer selected from the group consisting of: acrylic monomer having C₁₋₁₈ alkyl group; acrylic monomer having an acrylic acid, methacrylic acid or hydroxyl group; vinylpyrrolidone; and a mixture thereof.

8. The composition for transdermal absorption of claim 6, wherein the rubber-based adhesive is selected from the group consisting of natural rubber, styrene copolymer, polyisobutylene, polyisoprene, polybutadiene and a mixture thereof.

9. The composition for transdermal absorption of claim 1, which further comprises a polar aprotic organic solvent in an amount ranging from 0.1 to 20% by weight based on the total weight of the solid.

10. The composition for transdermal absorption of claim 9, wherein the polar aprotic organic solvent is selected from the group consisting of dimethylsulfoxide, dimethylacetamide, dimethylformamide, N-methylpyrrolidone, dimethylisosorbide, isosorbide and a mixture thereof.

11. The composition for transdermal absorption of claim 1, which further comprises a penetration enhancer selected from the group consisting of fatty acids, polyhydric alcohols, surfactants, fatty acid alcohol, fatty acid ester, polyoxyethylene alkylether, alkanolamine, alkanolamide and a mixture thereof in an amount ranging from 0.1 to 20% by weight based on the total weight of the solid.

12. The composition for transdermal absorption of claim 1, which further comprises a property controlling agent selected from the group consisting of polyvinylpyrrolidone, polyethyleneoxide, silicon oxide, aluminum oxide, titanium oxide and a mixture thereof in an amount ranging from 0.1 to 10% by weight based on the total weight of the solid.

13. A transdermal absorption formulation comprising a release paper; a polymeric matrix prepared from the composition for transdermal absorption of claim 1 and a supporting substrate stacked together in that order.

14. The transdermal absorption formulation of claim 13, wherein the polymeric matrix has a thickness ranging from 10 to 120 μm.

15. The transdermal absorption formulation of claim 13, wherein the release paper is a paper or a polymer film coated with silicon or a hydrofluorocarbon resin.

16. The transdermal absorption formulation of claim 13, wherein the supporting substrate is a film made from a material selected from the group consisting of polyolefin, polyester, polyurethane, aluminum and a mixture thereof, or a fabric or a nonwoven made from a material selected from the group consisting of polyolefin, polyester, nylon, cotton and a mixture thereof.

17. The transdermal absorption formulation of claim 13, which further comprises a supporting substrate adhesive layer having a thickness of 10 to 100 μm which is disposed between the supporting substrate and the polymeric matrix.

18. The transdermal absorption formulation of claim 17, wherein the supporting substrate adhesive layer comprises the polymeric adhesive and the adhesive performance improving agent in amounts of 70 to 99% by weight and 1 to 30% by weight, respectively, based on the total weight of the solid.

19. The transdermal absorption formulation of claim 13, which further comprises a skin adhesive layer having a thickness of 10 to 100 μm which is disposed between the release paper and the polymeric matrix.

20. The transdermal absorption formulation of claim 19, wherein the skin adhesive layer comprises the polymeric adhesive and the adhesive performance improving agent in an amount of 70 to 99% by weight and 1 to 30% by weight, respectively, based on the total weight of the solid.

21. The transdermal absorption formulation of claim 18, wherein the polymeric adhesive is acrylic adhesive or rubber-based polymeric adhesive, and the adhesive performance improving agent is selected from the group consisting of fatty acid ester, polyethyleneglycol, polyethyleneglycol-polypropyleneglycol copolymer, polyvinylpyrrolidone, an inorganic filler and a mixture thereof.

22. The transdermal absorption formulation of claim 21, wherein the acrylic adhesive is obtained by polymerizing a monomer selected from the group consisting of: acrylic monomer having C₁₋₁₈ alkyl group; acrylic monomer having an acrylic acid, methacrylic acid or hydroxyl group; vinylpyrrolidone; and a mixture thereof.

23. The transdermal absorption formulation of claim 21, wherein the rubber-based adhesive is selected from the group consisting of natural rubber, styrene copolymer, polyisobutylene, polyisoprene, polybutadiene and a mixture thereof.
